(19) United States
(12) Patent Application Publication
(10) Pub. No.: US 2006/0252749 A1
(43) Pub. Date: Nov. 9, 2006

(54) Lacosamide for add-on therapy of psychosis

(75) Inventor: Thomas Stohr, Monheim (DE)

Correspondence Address: HARNESS, DICKEY, & PIERCE, P.L.C, 7700 BONHOMME, STE 400, ST. LOUIS, MO, 63105, US

(73) Assignee: SRZ Properties, Inc., Wilmington, DE (US)

(21) Appl. No.: 11/342,140

(22) Filed: Jan. 27, 2006

Related U.S. Application Data
(60) Provisional application No. 60/647,410, filed on Jan. 28, 2005.

(51) Int. Cl. A61K 31/551; A61K 31/5415; A61K 31/496; A61K 31/519; A61K 31/445; A61K 31/165; A61K 31/137

(52) U.S. Cl. 514/220; 514/575; 514/616; 514/225.8; 514/254.05; 514/253.06; 514/317; 514/259.41; 514/649

(57) ABSTRACT

A therapeutic combination, useful in a co-therapy method for prevention, alleviation or treatment of psychosis, comprises a first agent and a second agent, wherein the first agent comprises at least one psychosis-treating compound and the second agent comprises at least one compound according to Formula (III): 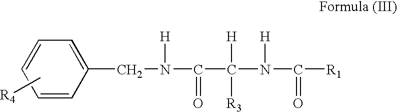
or a pharmaceutically acceptable salt thereof, wherein R₄ is one or more substituents independently selected from the group consisting of hydrogen, halo, alkyl, alkenyl, alkynyl, nitro, carboxy, formyl, carboxyamido, aryl, quaternary ammonium, haloalkyl, aryl alkanoyl, hydroxy, alkoxy, amino, alkylamino, dialkylamino, aryloxy, mercapto, alkylthio, alkylmercapto, and disulfide; R₃ is selected from the group consisting of hydrogen, alkyl, alkoxy, alkoxyalkyl, aryl, N-alkoxy-N-alkylamino, and N-alkoxyamino; and R₁ is alkyl.

This application claims priority of U.S. provisional application Ser. No. 60/647,410 filed on Jan. 28, 2005, the disclosure of which is incorporated herein by reference in its entirety.

### FIELD OF THE INVENTION

The present invention relates to the use of a class of peptide compounds that includes lacosamide for the prevention, alleviation or treatment of psychosis in add-on therapy.

### BACKGROUND OF THE INVENTION

Certain peptides are known to exhibit central nervous system (CNS) activity and are useful in the treatment of epilepsy and other CNS disorders. These peptides which are described in the U.S. Pat. No. 5,378,729 have the Formula (Ia): 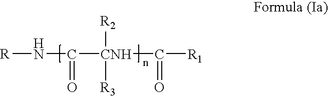
wherein:
R is hydrogen, lower alkyl, lower alkenyl, lower alkynyl, aryl, aryl lower alkyl, heterocyclic, heterocyclic lower alkyl, lower alkyl heterocyclic, lower cycloalkyl, lower cycloalkyl lower alkyl, and R is unsubstituted or is substituted with at least one electron withdrawing group or electron donating group;
R₁ is hydrogen or lower alkyl, lower alkenyl, lower alkynyl, aryl lower alkyl, aryl, heterocyclic lower alkyl, heterocyclic, lower cycloalkyl, lower cycloalkyl lower alkyl, each unsubstituted or substituted with an electron donating group or an electron withdrawing group;
R₂ and R₃ are independently hydrogen, lower alkyl, lower alkenyl, lower alkynyl, aryl lower alkyl, aryl, heterocyclic, heterocyclic lower alkyl, lower alkyl heterocyclic, lower cycloalkyl, lower cycloalkyl lower alkyl, or Z-Y wherein R₂ and R₃ may be unsubstituted or substituted with at least one electron withdrawing group or electron donating group;
Z is O, S, S(O)ₐ, NR₄, PR₄ or a chemical bond;
Y is hydrogen, lower alkyl, aryl, aryl lower alkyl, lower alkenyl, lower alkynyl, halo, heterocyclic, heterocyclic lower alkyl, and Y may be unsubstituted or substituted with an electron donating group or an electron withdrawing group, provided that when Y is halo, Z is a chemical bond, or
Z-Y taken together is NR₄NR₅R₇, NR₄OR₅, ONR₄R₇, OPR₄R₅, PR₄OR₅, SNR₄R₇, NR₄SR₇, SPR₄R₅ or PR₄SR₇, NR₄PR₅R₆, PR₄NR₅R₇, 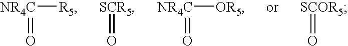
R₄, R₅ and R₆ are independently hydrogen, lower alkyl, aryl, aryl lower alkyl, lower alkenyl, or lower alkynyl, wherein R₄, R₅ and R₆ may be unsubstituted or substituted with an electron withdrawing group or an electron donating group;
R₇ is R₆ or COOR₈ or COR₈;
R₈ is hydrogen or lower alkyl, or aryl lower alkyl, and the aryl or alkyl group may be unsubstituted or substituted with an electron withdrawing group or an electron donating group;
n is 1-4; and
a is 1-3.

U.S. Pat. No. 5,773,475 also discloses additional compounds useful for treating CNS disorders. These compounds have the Formula (IIa): 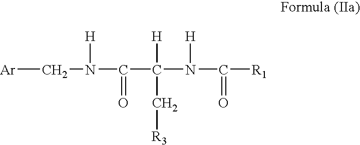
wherein Ar is aryl which is unsubstituted or substituted with halo; R₃ is lower alkoxy; and R₁ is methyl.

WO 02/074297 relates to the use of a compound according to Formula (IIa) wherein Ar is phenyl which may be substituted by at least one halo, R₃ is lower alkoxy containing 1-3 carbon atoms and R₁ is methyl for the preparation of pharmaceutical compositions useful for the treatment of allodynia related to peripheral neuropathic pain.

WO 02/074784 relates to the use of a compound having Formula (Ia) or Formula (IIa) showing antinociceptive properties for treating different types and symptoms of acute and chronic pain, especially non neuropathic inflammatory pain, e.g., rheumatoid arthritic pain or secondary inflammatory osteo-arthritic pain.

Psychosis, in particular schizophrenia, is a state of mental impairment marked by the following symptoms:

Distorted Perceptions of Reality

People with pyschosis may have distorted perceptions of reality that are strikingly different from the reality seen and shared by others around them.

Hallucinations and Illusions

Hallucinations are perceptions that occur without connection to an appropriate source. Hallucinations can occur in any sensory form—auditory (sound), visual (sight), tactile (touch), gustatory (taste), and olfactory (smell). Auditory hallucinations, particularly the experience of hearing voices that other people do not hear, are a common and often prominent feature of psychosis.

Delusions

Delusions are false personal beliefs that are not subject to reason or contradictory evidence and are not explained by a person's usual cultural concepts.

Disordered Thinking

Psychosis often affects a person's ability to “think straight.” Thoughts may come and go rapidly; the person may not be able to concentrate on one thought for very long and may be easily distracted, unable to focus attention. Disordered thinking is classified largely by its effect on speech and writing.

Psychosis is considered to be a symptom of severe mental illness, but not a diagnosis in itself. Although it is not exclusively linked to any particular psychological or physical state, it is particularly associated with schizophrenia, bipolar disorder and severe clinical depression. There are also several physical circumstances that can induce a psychotic state, including electrolyte disorder, urinary tract infections in the elderly, pain syndromes, drug toxicity, and drug withdrawal (especially alcohol, barbiturates, and sometimes benzodiazepines) as well as infections of or injuries to the brain (these psychoses are now more commonly referred to as organic mental disorders.)

Chronic psychological stress is also known to cause psychotic states, though the exact mechanism is uncertain. Short-lived psychosis triggered by stress is known as brief reactive psychosis.

There are also some non-psychiatric conditions which are linked particularly to psychosis, which may include brain tumor, dementia with Lewy bodies, hypoglycemia, intoxication, multiple sclerosis, systemic lupus erythematosus, or/and sarcoidosis.

Psychosis is a common condition in schizophrenia, marked by the typical, above mentioned symptoms. In addition, people with schizophrenia often show “blunted” or “flat” affect. This refers to a severe reduction in emotional expressiveness.

Schizophrenia is a chronic, severe, and disabling brain disease found all over the world. Approximately 1% of the population develops schizophrenia during their lifetime. The severity of the symptoms and long-lasting, chronic pattern of schizophrenia often cause a high degree of disability. People with schizophrenia have a higher rate of suicide than the general population. Approximately 10% of people with schizophrenia (especially younger adult males) commit suicide. The first signs of schizophrenia often appear as confusing, or even shocking, changes in behavior. This sudden onset of severe psychotic symptoms is referred to as an “acute” phase of schizophrenia.

Some people have only one such psychotic episode; others have many episodes during a lifetime, but lead relatively normal lives during the interim periods. However, the individual with “chronic” schizophrenia, or a continuous or recurring pattern of illness, often does not fully recover normal functioning and typically requires long-term treatment, generally including medication, to control the symptoms.

There is no known single cause of schizophrenia. Many diseases, such as heart disease, result from interplay of genetic, behavioral, and other factors; and this may be the case for schizophrenia as well. It has long been known that schizophrenia runs in families. Scientists are studying genetic factors in schizophrenia. It appears likely that multiple genes are involved in creating a predisposition to develop the disorder. In addition, factors such as prenatal difficulties like intrauterine starvation or viral infections, perinatal complications, and various nonspecific stressors, seem to influence the development of schizophrenia. However, it is not yet understood how the genetic predisposition is transmitted, and it cannot yet be accurately predicted whether a given person will or will not develop the disorder.

Basic knowledge about brain chemistry and its link to schizophrenia is expanding rapidly. It is likely, although not yet certain, that the disorder is associated with some imbalance of the complex, interrelated chemical systems of the brain, perhaps involving the neurotransmitters dopamine and glutamate.

Many studies of people with schizophrenia have found abnormalities in brain structure (for example, enlargement of the fluid-filled cavities, called the ventricles, in the interior of the brain, and decreased size of certain brain regions) or function (for example, decreased metabolic activity in certain brain regions). It should be emphasized that these abnormalities are quite subtle and are not characteristic of all people with schizophrenia, nor do they occur only in individuals with this illness. Microscopic studies of brain tissue after death have also shown small changes in distribution or number of brain cells in people with schizophrenia. It appears that many (but probably not all) of these changes are present before an individual becomes ill, and schizophrenia may be, in part, a disorder in development of the brain.

Available treatments can relieve many symptoms, but most people with schizophrenia continue to suffer some symptoms throughout their lives; it has been estimated that no more than one in five individuals recovers completely. Medications and other treatments for schizophrenia can help reduce and control the distressing symptoms of the illness. However, some people are not greatly helped by available treatments or may prematurely discontinue treatment because of unpleasant side effects. Since schizophrenia may not be a single condition and its causes are not yet known, current treatment methods are based on both clinical research and experience. These approaches are chosen on the basis of their ability to reduce the symptoms of schizophrenia and to lessen the chances that symptoms will return.

Antipsychotic medications have been available since the mid-1950s but they do not “cure” the underlying disease such as schizophrenia or ensure that there will be no further psychotic episodes. Clinically effective antipsychotic agents include tricyclic phenothiazines, thioxanthenes, and dibenzeipines, as well as butyrophenones and congeners, other heterocyclics and experimental benzamides. Virtually all of these drugs block D₂-dopamine receptors and reduce dopamine neurotransmission in the forebrain; some of these drugs, in particular the atypical antipsychotics, also interact with D₁- and D₄-dopaminergic, 5-HT_{2A}- and 5-HT_{2C}-serotonergic and alpha-adrenergic receptors (Goodman & Gilman's The Pharmacological Basis of Therapeutics, 10th edition, McGraw-Hill, 2001: Summary of chapter 20 (p. 485)). Antagonism of dopamine-mediated synaptic neurotransmission is an important action of antipsychotic drugs (same document, p. 493, left column, 2nd para).

A number of new antipsychotic agents (the so-called “atypical antipsychotics”) have been introduced since 1990. The first of these, clozapine, has been shown to be more effective than other antipsychotics, although the possibility of severe side effects—in particular, a condition called agranulocytosis (loss of the white blood cells that fight infection)—requires that patients be monitored with blood tests every one or two weeks. Even newer antipsychotic agents, such as risperidone and olanzapine are safer than the older drugs or clozapine, and they also may be better tolerated. They may or may not treat the illness as well as clozapine, however.

Antipsychotic agents are often very effective in treating certain symptoms of schizophrenia, particularly hallucinations and delusions; unfortunately, the drugs may not be as helpful with other symptoms, such as reduced motivation and emotional expressiveness.

Current limitations of antipsychotic agent therapy of psychosis, especially psychosis associated with schizophrenia, include limited efficacy, side effects and delayed onset of action. Valproate was shown to enhance the onset of action of olanzapine or risperidone (Casey et al., Neuropsychopharmacology 28:182ff, 2003). Furthermore, another double-blind placebo controlled clinical trial demonstrated that lamotrigine, when added to clozapine, is beneficial for the treatment of treatment-resistant schizophrenia (Tihohen et al., Biol. Psychiatr. 54:1241ff, 2003). It is estimated that currently up to 50% of hospitalized schizophrenic patients receive adjunct therapy with anti-epileptic drugs (Citrome L., Psychopharmacology Bull. 37(S2):p74ff, 2003) especially for enhancing the onset of action, for controlling the positive symptoms, e.g., hallucinations, the affective symptoms, e.g., depression, as well as the cognitive symptoms, e.g., attention deficit.

However, as described in Citrome L., Psychopharmacology Bull. 37(S2):p74ff, 2003, data on the use of anticonvulsants for add-on therapy to antipsychotics are inconsistent. Valproate and lamotrigine were shown to preferentially reduce the positive symptoms of schizophrenia (same document, p. 86, last para). A possible locus of action of valproate was said to be direct effects on voltage gated sodium channels (same document, page 78, 2nd para). However, the use of the anticonvulsant carbamazepine as add-on to the antipsychotic agent haloperidol was associated with a worse clinical outcome compared with antipsychotic monotherapy (same document, page 80, first para). In another study on the use of carbamazepine there was a failure to detect significant improvement on the total BPRS (brief psychiatric rating scale). Adjunctive use of lamotrigine in addition to clozapine resulted in improvement of positive, not negative symptoms of schizophrenia (same document, page 85, 1st para). Reports on the use of other anticonvulsants in schizophrenia report general worsening of psychosis with gabapentin or deterioration in both positive and negative symptoms of schizophrenia with topiramate (same document, page 85, 2nd para).

The reported differences in the efficacy of anticonvulsants as add-on therapeutics to antipsychotics might be due to their different modes of action. In contrast to antipsychotic agents that share a common antagonistic action on dopamine receptors, current anticonvulsants are believed to work through diverse mechanisms of action. These mechanisms of action are, e.g., altering neuronal impulse propagation via interaction with voltage-gated sodium, calcium, or potassium channels or affecting neural transmission by either potentiating inhibitory GABA systems or by inhibition of excitatory glutamate systems.

In contrast to current anticonvulsants, lacosamide potentially has a unique but yet unknown molecular mechanism of action. Lacosamide does not directly interact with a variety of known GABA receptor subtypes, nor does it directly interact with a variety of known glutamate receptor subtypes. In particular, lacosamide does not inhibit voltage-gated sodium or calcium channels and does not potentiate potassium currents.

### SUMMARY OF THE INVENTION

The present inventors have discovered a therapeutic combination comprising a first agent and a second agent, wherein the first agent comprises at least one psychosis-treating compound and the second agent comprises at least one compound according to Formula (III): 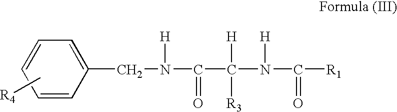
or a pharmaceutically acceptable salt thereof, wherein
R₄ is one or more substituents independently selected from the group consisting of hydrogen, halo, alkyl, alkenyl, alkynyl, nitro, carboxy, formyl, carboxyamido, aryl, quaternary ammonium, haloalkyl, aryl alkanoyl, hydroxy, alkoxy, amino, alkylamino, dialkylamino, aryloxy, mercapto, alkylthio, alkylmercapto, and disulfide;
R₃ is selected from the group consisting of hydrogen, alkyl, alkoxy, alkoxyalkyl, aryl, N-alkoxy-N-alkylamino, and N-alkoxyamino; and
R₁ is alkyl.

The present inventors have further discovered a co-therapy method for the prevention, alleviation or treatment of psychosis, in particular psychosis associated with schizophrenia, comprising co-administration of a first agent and a second agent, wherein the first agent comprises at least one psychosis-treating compound, and the second agent comprises at least one compound of Formula (III).

Other compounds generic to Formula (III), for example compounds of Formula (Ib) as defined below, can be substituted for the Formula (III) compound in combinations and methods of the invention.

### DETAILED DESCRIPTION

The use of compounds of Formula (III) for treatment of psychosis, in particular psychosis associated with schizophrenia, in an add-on therapy to at least one antipsychotic agent has not been reported. Thus, the present invention concerns the use of compound(s) of Formula (III) for the prevention, alleviation or treatment of psychosis, in particular in the course of schizophrenia, in an add-on therapy to at least one psychosis-treating compound, or for the preparation of a pharmaceutical composition useful in such prevention, alleviation or treatment.

In accordance with the invention, a study reported in the Example herein found that the application of (R)-2-acetamide-N-benzyl-3-methoxypropionamide (international non-proprietary name lacosamide, also formerly called harkoseride or SPM 927) alone had no significant effect on prepulse inhibition in mice, which is a model of predictive validity for psychosis, particularly for cognitive symptoms. Surprisingly, lacosamide potentiated the increase of prepulse inhibition by the antipsychotic agent clozapine, when the lacosamide and clozapine were administered together.

Prepulse inhibition in mice is a model of predictive value for psychosis associated with, for example, schizophrenia, bipolar disorder, autism, Alzheimer's disease, attention deficit hyperactivity disorder, drug abuse, alcohol abuse, affective disorders, dyskinesias and related disorders, dementia, mental retardation, polydipsia/hyponatremia, severe personality disorder, intractable chronic insomnia, Huntington's disease, Tourette's syndrome, Parkinson's disease, and dopaminergic therapy of Parkinson's disease.

Considering the modes of action of anticonvulsants, the variability of results in add-on therapy of anticonvulsants with antipsychotics, the difference in mode of action of valproate, the only anticonvulsant with established usefulness in add-on therapy with antipsychotics, as compared to lacosamide, and the lack of mention of anticonvulsants in an article on add-on therapy of cognitive symptoms of schizophrenia (Jann M W, Pharmacotherapy 2004, 24(12):1759-1783, incorporated herein by reference but not admitted to be prior art to the present invention), a person skilled in the art would not have expected the usefulness of lacosamide in add-on therapy of psychosis with at least one antipsychotic.

The term “psychosis-treating” applied to a compound herein means that the compound is capable of producing a therapeutic effect on a psychotic condition or a symptom or underlying cause of such condition in a subject, but is not itself an anticonvulsant. “Therapeutic” effects include prevention, partial or total relief of symptoms, remediation, amelioration, alleviation, correction and cure of the condition. In one embodiment, the psychosis-treating compound is a dopamine antagonist or partial agonist having antipsychotic properties. Typically, such a compound is a D₂ antagonist or partial agonist, but in some cases can also interact with D₁ or D₄ receptors, as in the case of certain “atypical” antipsychotics.

The term “therapeutic combination” refers to a plurality of agents that, when administered to a subject together or separately, are co-active in bringing therapeutic benefit to the subject. Such administration is referred to as “combination therapy,” “co-therapy,” “adjunctive therapy” or “add-on therapy.” For example, one agent can potentiate or enhance the therapeutic effect of another, or reduce an adverse side effect of another, or one or more agents can be effectively administered at a lower dose than when used alone, or can provide greater therapeutic benefit than when used alone, or can complementarily address different aspects, symptoms or etiological factors of a disease or condition.

The term “patient” or “subject” refers to a warm-blooded animal, and preferably a mammal, such as, for example, a cat, dog, horse, cow, pig, mouse, rat or primate, including a human. The preferred patient is a human.

As indicated above, the present invention provides a therapeutic combination comprising a first agent and a second agent, wherein the first agent comprises at least one psychosis-treating compound and the second agent comprises at least one compound according to Formula III.

The present invention also provides a co-therapy method for the prevention, alleviation or treatment of psychosis, in particular psychosis associated with schizophrenia, comprising administering to a subject a first amount of a first agent comprising at least one psychosis-treating compound and a second amount of a second agent comprising at least one compound according to Formula (III), wherein the first amount and second amount together provide a therapeutically effective combination of the first agent and second agent. The co-therapy method can have one or more of a number of objectives and results, including without limitation to increase the efficacy, decrease the side effects, or enhance the onset of action of the psychosis-treating compound, for example.

Co-administration of a first and a second agent, as in the co-therapy method of the invention, comprises administration of the agents in amounts sufficient to achieve or maintain therapeutically effective concentrations, e.g., plasma concentrations, in the subject in need thereof. Co-administration can comprise one or both of simultaneous and subsequent (i.e., sequential) administration. Simultaneous administration can comprise administration of the agents as a single composition or as different compositions (see below) “at the same time” within a treatment period. Sequential administration can comprise administration of the agents at different times, for example “at intervals” within a treatment period.

Administration “at the same time” includes administration of the first and second agents literally “at the same time,” but also includes administration directly one after the other, in any order. Administration “at intervals” includes administration of the at least one psychosis-treating compound and the compound(s) of Formula (III) at different times, separated for example by an interval of about 1 h, about 6 h, about 12 h, about 1 day, or about 1 month at the maximum.

The at least one psychosis-treating compound and the at least one compound of Formula (III) may be formulated in one pharmaceutical preparation (single dose form) for administration at the same time or may be formulated in two distinct preparations (separate dose forms) for administration at the same time or sequentially, as described for various embodiments below, for example. The two distinct preparations in the separate dose forms may be administered by the same route or by different routes.

Separate dose forms can optionally be co-packaged, for example in a single container or in a plurality of containers within a single outer package, or co-presented in separate packaging (“common presentation”). As an example of co-packaging or common presentation, a kit is contemplated comprising, in separate containers, the at least one psychosis-treating compound and the at least one compound of Formula (III). In another example, the at least one psychosis-treating compound and the at least one compound of Formula (III) are separately packaged and available for sale independently of one another, but are co-marketed or co-promoted for use according to the invention. The separate dose forms may also be presented to a subject separately and independently, for use according to the invention.

Depending on the dosage forms, which may be identical or different, e.g., fast release dosage forms, controlled release dosage forms or depot forms, the at least one psychosis-treating compound and the at least one compound of Formula (III) may be administered on the same or on different schedules, for example on a daily, weekly or monthly basis. Therefore, the administration interval in a co-therapy method of the invention may depend on the administration schedules or on the dosage forms.

As discussed above, the first agent comprises at least one psychosis-treating compound. The at least one psychosis-treating compound can be any compound known in the art for prevention, alleviation or treatment of psychosis, including without limitation those illustratively tabulated below:

| Psychosis-treating | | | |
|---|---|---|---|
| compound [CAS Ref.] | Mode of action | Illustrative dose | Reference |
| Amisulpride | Dopamine D2/D3 | 200-1200 mg/day | http://en.wikipedia.org/wiki/Amisulpride |
| [71675-85-9] | antagonist | | |
| Aripiprazole | Dopamine D2 partial | The recommended | http://www.healthyplace.com/Communities/Thought_Disorders/ |
| [129722-12-9] | agonist | starting and target | schizo/medications/aripiprazole.asp#DOSAGE |
| | | dose is 10 or 15 mg/day | |
| | | administered | |
| | | on a once-a-day | |
| | | schedule without | |
| | | regard to meals. | |
| Biriperone | Dopamine antagonist | 3-4.5 mg/day p.o. | |
| [42021-34-1] | | | |
| Bromperidol | Dopamine D2 | 3-18 mg/day | http://chrom.tutms.tut.ac.jp/JINNO/DRUGDATA/46bromperidol.html |
| [10457-90-6] | antagonist | | |
| Carpipramine | Dopamine D2 | | |
| [5942-95-0; 7075-03-8] | antagonist | | |
| Clocapramine | Serotonin antagonist; | 30-150 mg/day | http://chrom.tutms.tut.ac.jp/JINNO/DRUGDATA/ |
| [28058-62-0; | targets dopamine D2 | | 57clocapramine.html |
| 47739-98-0] | receptor | | |
| Clorotepine | Dopamine antagonist | | |
| [13448-22-1] | | | |
| Clozapine [5786-21-0] | Dopamine antagonist | Achieve a target dose | http://www.rxlist.com/cgi/generic3/clozapine_ids.htm |
| | | of 300-450 mg/day | http://www.nlm.nih.gov/medlineplus/druginfo/uspdi/202157.html |
| | | by the end of 2 | |
| | | weeks. Adults - At | |
| | | first, 12.5 mg once or | |
| | | twice a day. | |
| | | However, the dose | |
| | | usually is not more | |
| | | than 900 mg a day. | |
| Isofloxythepin | Dopamine antagonist | 5 mg/kg orally | Psychopharmacology (Berl.) |
| [106819-39-0; | | | 1982; 76(4): 381-4. |
| 106819-41-4; | | | |
| 70931-18-9] | | | |
| Melperone | Dopamine D2 | 300 mg/day | Sumiyoshi: Schizophrenia |
| [1622-79-3; 3575-80-2] | antagonist | | Research 62(2): 65 |
| Mosapramine | Dopamine D2 | 6.25-25 mg/day | |
| [89419-40-9; | antagonist | | |
| 98043-60-8] | | | |
| Nemonapride | Dopamine D2/D3 | 9-36 mg/day | |
| [75272-39-8] | antagonist | | |
| Olanzapine | Dopamine D2 | 10 mg/day | http://www.mentalhealth.com/drug/p30-o02.html |
| [132539-06-1] | antagonist | | |
| Penfluridol [26864-56-2] | Dopamine antagonist | 60-80 mg once | J. Clin. Pharmacol., |
| | (D1 and D2) | weekly | Apr. 1, 1977; 17(4): 252-258. |
| Perospirone | Dopamine D2 | 8-48 mg/day | |
| [129273-38-7; | antagonist | | |
| 150915-41-6] | | | |
| Pimozide [2062-78-4] | Dopamine D2 | The average | http://home.intekom.com/pharm/janssen/orap-t.html |
| | antagonist | maintenance dose is | |
| | | 6 mg daily with the | |
| | | usual range of 2 to | |
| | | 12 mg per day | |
| Pipotiazine palmitate | Dopamine antagonist; | To be administered | http://www.mentalhealth.com/drug/p30-p03.html#Head_8 |
| [37517-26-3; | targets dopamine D2 | as an i.m. injection | |
| 39860-99-6] | receptor | only; it is frequently | |
| | | possible to achieve | |
| | | adequate control with | |
| | | a dosage between 75 | |
| | | and 150 mg | |
| | | administered every 4 | |
| | | weeks | |
| Quetiapine fumarate | Dopamine D2 | Varies | University of Alberta website |
| [111974-69-7; | antagonist | | called ‘DrugBank’; |
| 111974-72-2] | | | http://www.rxlist.com/cgi/generic2/quetiap_ids.htm#D |
| Risperidone | Dopamine D2 | 6 mg/day | http://ajp.psychiatryonline.org/cgi/reprint/157/7/1178-a |
| [106266-06-2] | antagonist | Risperdal Consta: | |
| | | 25-75 mg/day i.m. | |
| | | Risperdal Instasolv: | http://www.rxlist.com/cgi/generic/risperid_cp.htm#CP |
| | | mean modal dose 4.1 mg/day | |
| Sulpiride [15676-16-1] | Dopamine D2 | 600-800 mg/day | http://www.mentalhealth.com/drug/p30-s06.html |
| | antagonist | i.m.; or 200-3200 mg/day | |
| | | in 2 or 3 | |
| | | divided doses orally | |
| Sultopride [53583-79-2] | Dopamine D2 | | |
| | antagonist | | |
| Tiapride [51012-32-9] | Dopamine D2 | Usual final dose is | http://www.spisdemo.com/RX/ |
| | antagonist | 200-300 mg/day, | RXDisplay.aspx?qsRXID=RX22030204&qsSection=5 |
| | | continued for 1-2 | |
| | | months | |
| Timiperone | Dopamine D2 | 4-8 mg/day | |
| [57648-21-2] | antagonist | | |
| Ziprasidone | Dopamine D2 | Varies | http://www.geodon.com/hcp_home.asp |
| [122883-93-6; | antagonist | | |
| 146939-27-7] | | | |
| Zotepine [26615-21-4] | Dopamine antagonist | 75-300 mg/day | http://www.ukmi.nhs.uk/NewMaterial/html/docs/zotepine.pdf |
| Zuclopenthixol | Dopamine D2 | The usual therapeutic | http://www.mentalhealth.com/drug/p30-z03.html#Head_8 |
| [53772-83-1; 982-24-1] | antagonist | range is 20-60 mg/day | |
| Zuclopenthixol acetate | Dopamine D2 | 50-150 mg i.m., | http://www.vhpharmsci.com/PDTM/Monographs/ |
| [85721-05-7] | antagonist | repeated if necessary | zuclopenthixol%20acetate.htm |
| | | every 2-3 days; | |
| | | maximum cumulative | |
| | | dose should not | |
| | | exceed 400 mg and | |
| | | number of injections | |
| | | should not exceed 4 | |
| Zuclopenthixol | Dopamine D2 | The minimum | Pharmacopsychiatry 1994 |
| decanoate | antagonist | effective dose of | May; 27(3): 119-123 |
| [64053-00-5] | | zuclopenthixol was | |
| | | 200 mg/2 weeks | |
| | | (range 60-400), with | |
| | | a serum concentration | |
| | | of 22 nmol/l | |
| | | (7.1-69.7). | |

In one embodiment, the first agent comprises at least one compound selected from the group consisting of tricyclic antipsychotics, phenothiazines, thioxanthenes, butyrophenones, dihydroindolones, and dibenzoxazepines. Examples of compounds suitable for use in the first agent include, without limitation, clozapine, risperidone, aripiprazole, quetiapine, olanzapine, ziprasidone, sulpiride, amisulpride and zotepine. And most preferably, the at least one psychosis-treating compound is clozapine.

As discussed above, the second agent comprises at least one compound according to Formula (III).

Typically, alkyl, alkoxy, alkenyl and alkynyl groups in a compound of Formula (III) are lower alkyl, alkoxy, alkenyl and alkynyl groups having no more than 6, more typically no more than 3, carbon atoms.

In one embodiment, R₄ in a compound of Formula (III) is one or more hydrogen or halo, more particularly fluoro, substituents.

In one embodiment, R₃ in a compound of Formula (III) is lower-alkoxy-lower-alkyl, phenyl, N-lower-alkoxy-N-lower-alkylamino or N-lower-alkoxyamino.

In one embodiment, R₁ in a compound of Formula (III) is lower alkyl with 1-3 carbon atoms.

In a particular embodiment, R₄ is hydrogen or fluoro; R₃ is selected from the group consisting of methoxymethyl, phenyl, N-methoxy-N-methylamino and N-methoxyamino; and R₁ is methyl.

In various embodiments, compounds suitable for use in the second agent are:
(R)-2-acetamido-N-benzyl-3-methoxy-propionamide;
(R)-2-acetamido-N-benzyl-3-ethoxy-propionamide;
O-methyl-N-acetyl-D-serine-m-fluorobenzylamide;
O-methyl-N-acetyl-D-serine-p-fluorobenzylamide;
N-acetyl-D-phenylglycinebenzylamide;
D-1,2-(N,O-dimethylhydroxylamino)-2-acetamide acetic acid benzylamide; and
D-1,2-(O-methylhydroxylamino)-2-acetamide acetic acid benzylamide.

In one embodiment, a compound suitable for use in the second agent has the Formula (IIb): 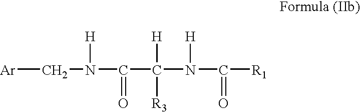
wherein Ar is aryl, especially phenyl, which is unsubstituted or substituted with at least one halo; R₃ is —CH₂-Q, wherein Q is lower alkoxy; and R₁ is lower alkyl, especially methyl. The most preferred R₁ is CH₃. The most preferred R₃ is CH₂-Q, wherein Q is methoxy.

In a particular embodiment, the second agent comprises (R)-2-acetamido-N-benzyl-3-methoxypropionamide (lacosamide).

Preferably the at least one compound of Formula (III), for example lacosamide, is substantially enantiopure. As used herein, the term “substantially enantiopure” means having at least 88%, preferably at 90%, more preferably at least 95, 96, 97, 98, or 99% enantiomeric purity.

In yet another embodiment, the first agent comprises one or more of amisulpride, aripiprazole, biriperone, bromperidol, carpipramine, clocapramine, clorotepine, clozapine, isofloxythepin, melperone, mosapramine, nemonapride, olanzapine, penfluridol, perospirone, pimozide, pipotiazine palmitate, quetiapine fumarate, risperidone, sulpiride, sultopride, tiapride, timiperone, ziprasidone, zotepine, zuclopenthixol, zuclopenthixol acetate and zuclopenthixol decanoate, and the second agent comprises lacosamide.

Preferably, the first agent comprises one or more of clozapine, risperidone, aripiprazole, quetiapine, olanzapine, ziprasidone, sulpiride, amisulpride and zotepine, and the second agent comprises lacosamide. Most preferably, the first agent comprises clozapine and the second agent comprises lacosamide.

In a particular embodiment, the compounds of Formula (III) are used for the preparation of a pharmaceutical composition comprising at least one psychosis-treating compound and at least one compound of Formula (III).

The present invention is also directed to a pharmaceutical composition comprising at least one compound according to Formula (III) useful for the prevention, alleviation or/and treatment of psychosis in an add-on therapy to at least one psychosis-treating compound.

In yet another particular embodiment of the present invention, a commercially available composition of a psychosis-treating compound may be administered to a subject in need thereof. Therefore, in this embodiment, the compounds of Formula (III) are used for the preparation of a pharmaceutical composition comprising at least one compound according to Formula (III) and not comprising a psychosis-treating compound.

The co-therapy method according to the present invention may comprise the preparation of a pharmaceutical composition for administration of the at least one compound of Formula (III) and the at least one psychosis-treating compound at the same time or at intervals, as defined above.

Although the invention is described herein with particular reference to compounds of Formula (III), a compound useful according to the invention can have the more general Formula (Ib) 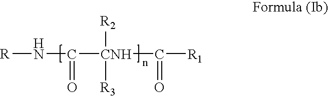
wherein:
R is hydrogen, lower alkyl, lower alkenyl, lower alkynyl, aryl, aryl lower alkyl, heterocyclic, heterocyclic lower alkyl, lower alkyl heterocyclic, lower cycloalkyl or lower cycloalkyl lower alkyl, and R is unsubstituted or is substituted with at least one electron withdrawing group, and/or at least one electron donating group;
R₁ is hydrogen or lower alkyl, lower alkenyl, lower alkynyl, aryl lower alkyl, aryl, heterocyclic lower alkyl, lower alkyl heterocyclic, heterocyclic, lower cycloalkyl, lower cycloalkyl lower alkyl, each unsubstituted or substituted with at least one electron donating group and/or at least one electron withdrawing group;
R₂ and R₃ are independently hydrogen, lower alkyl, lower alkenyl, lower alkynyl, aryl lower alkyl, aryl, halo, heterocyclic, heterocyclic lower alkyl, lower alkyl heterocyclic, lower cycloalkyl, lower cycloalkyl lower alkyl, or Z-Y wherein R₂ and R₃ may be unsubstituted or substituted with at least one electron withdrawing group and/or at least one electron donating group;
Z is O, S, S(O)ₐ, NR₄, NR′₆, PR₄ or a chemical bond;
Y is hydrogen, lower alkyl, aryl, aryl lower alkyl, lower alkenyl, lower alkynyl, halo, heterocyclic, heterocyclic lower alkyl, lower alkyl heterocyclic and Y may be unsubstituted or substituted with at least one electron donating group and/or at least one electron withdrawing group, provided that when Y is halo, Z is a chemical bond, or Z-Y taken together is NR₄NR₅R₇, NR₄OR₅, ONR₄R₇, OPR₄R₅, PR₄OR₅, SNR₄R₇, NR₄SR₇, SPR₄R₅, PR₄SR₇, NR₄PR₅R₆, PR4NR5R7, N⁺R₅R₆R₇, 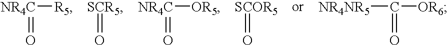
R′₆ is hydrogen, lower alkyl, lower alkenyl, or lower alkenyl which may be unsubstituted or substituted with at least one electron withdrawing group or/and at least one electron donating group;
R₄, R₅ and R₆ are independently hydrogen, lower alkyl, aryl, aryl lower alkyl, lower alkenyl, or lower alkynyl, wherein R₄, R₅ and R₆ may independently be unsubstituted or substituted with at least one electron withdrawing group or/and at least one electron donating group;
R₇ is R₆ or COOR₈ or COR₈, which R₇ may be unsubstituted or substituted with at least one electron withdrawing group or/and at least one electron donating group;
R₈ is hydrogen or lower alkyl, or aryl lower alkyl, and the aryl or alkyl group may be unsubstituted or substituted with at least one electron withdrawing group or/and at least one electron donating group;
n is 1-4; and
a is 1-3.

The term “alkyl” (alone or in combination with another term(s)) means a straight- or branched-chain saturated hydrocarbyl substituent typically containing from 1 to about 20 carbon atoms, more typically from 1 to about 8 carbon atoms, and even more typically from 1 to about 6 carbon atoms.

The term “lower alkyl” refers to an alkyl substituent containing from 1 to 6 carbon atoms, especially 1 to 3 carbon atoms, and may be straight-chain or branched. Examples include methyl, ethyl, propyl, isopropyl, butyl, isobutyl, tertiary butyl, pentyl, hexyl, and the like, and isomers thereof.

The term “alkenyl” (alone or in combination with another term(s)) means a straight- or branched-chain hydrocarbyl substituent containing one or more double bonds and typically from 2 to about 20 carbon atoms, more typically from 2 to about 8 carbon atoms, and even more typically from 2 to about 6 carbon atoms. The alkenyl groups, where asymmetric, can have cis or trans configuration.

The term “lower alkenyl” refers to an alkenyl group containing from 2 to 6 carbon atoms and may be straight-chained or branched and in the Z or E form. Examples include vinyl, propenyl, 1-butenyl, isobutenyl, 2-butenyl, 1-pentenyl, (Z)-2-pentenyl, (E)-2-pentenyl, (Z)-4-methyl-2-pentenyl, (E)-4-methyl-2-pentenyl, pentadienyl, e.g., 1, 3 or 2,4-pentadienyl, and the like.

The term “alkynyl” (alone or in combination with another term(s)) means a straight- or branched-chain hydrocarbyl substituent containing one or more triple bonds and typically from 2 to about 20 carbon atoms, more typically from 2 to about 8 carbon atoms, and even more typically from 2 to about 6 carbon atoms.

The term “lower alkynyl” refers to an alkynyl group containing 2 to 6 carbon atoms and may be straight-chained or branched. It includes such groups as ethynyl, propynyl, 1-butynyl, 2-butynyl, 1-pentynyl, 2-pentynyl, 3-methyl-1-pentynyl, 3-pentynyl, 1-hexynyl, 2-hexynyl, 3-hexynyl and the like.

The term “lower cycloalkyl” or “cycloalkyl” when used alone or in combination is a completely or partially saturated alicyclic hydrocarbyl group containing from about 3 to about 18 ring carbon atoms. The cycloalkyl groups may be monocyclic or polycyclic. Examples include cyclopropyl, cyclobutyl, cyclopentyl, cyclohexyl, cycloheptyl, cyclooctyl, cyclodecyl, cyclohexenyl, cyclopentenyl, cyclooctenyl, cycloheptenyl, decalinyl, hydroindanyl, indanyl, fenchyl, pinenyl, adamantyl, and the like. Cycloalkyl includes the cis or trans forms. Cycloalkyl groups may be unsubstituted or mono- or polysubstituted with electron withdrawing or/and electron donating groups as described below. Furthermore, the substituents may either be in endo or exo positions in bridged bicyclic systems.

The term “alkoxy” (alone or in combination with another term(s)) means an alkylether substituent, i.e., —O-alkyl.

The term “lower alkoxy” means alkoxy containing from 1 to 6 carbon atoms, especially 1 to 3 carbon atoms, and may be straight-chain or branched. Examples include methoxy, ethoxy, propoxy, butoxy, isobutoxy, tert-butoxy, pentoxy, hexoxy and the like.

The term “aryl”, when used alone or in combination, refers to an aromatic group which contains from about 6 to about 18 ring carbon atoms and includes polynuclear aromatics. These aryl groups may be monocyclic or polycyclic, and optionally fused. A polynuclear aromatic group as used herein encompasses bicyclic and tricyclic fused aromatic ring systems containing from about 10 to about 18 ring carbon atoms. Aryl groups include phenyl, and the polynuclear aromatics e.g., naphthyl, anthracenyl, phenanthrenyl, azulenyl and the like. The aryl group also includes groups such as ferrocenyl. Aryl groups may be unsubstituted or mono- or polysubstituted with electron withdrawing or/and electron donating groups as described below.

“Aryl lower alkyl” groups include, for example, benzyl, phenylethyl, phenylpropyl, phenylisopropyl, phenylbutyl, diphenylmethyl, 1,1-diphenylethyl, 1,2-diphenylethyl, and the like.

The term “monosubstituted amino” (alone or in combination with another term(s)) means an amino substituent wherein one of the hydrogen radicals is replaced by a non-hydrogen substituent. The term “disubstituted amino” (alone or in combination with another term(s)) means an amino substituent wherein both of the hydrogen atoms are replaced by non-hydrogen substituents, which may be identical or different.

The term “halo” or “halogen” includes fluoro, chloro, bromo, and iodo.

The term “carbalkoxy” refers to —CO—O-alkyl, wherein alkyl may be lower alkyl as defined above.

The prefix “halo” indicates that the substituent to which the prefix is attached is substituted with one or more independently selected halogen radicals. For example, haloalkyl means an alkyl substituent wherein at least one hydrogen radical is replaced with a halogen radical. Examples of haloalkyls include chloromethyl, 1-bromoethyl, fluoromethyl, difluoromethyl, trifluoromethyl, 1,1,1-trifluoroethyl, and the like. Illustrating further, “haloalkoxy” means an alkoxy substituent wherein at least one hydrogen radical is replaced by a halogen radical. Examples of haloalkoxy substituents include chloromethoxy, 1-bromoethoxy, fluoromethoxy, difluoromethoxy, trifluoromethoxy (also known as “perfluoromethyloxy”), 1,1,1,-trifluoroethoxy, and the like. It should be recognized that if a substituent is substituted by more than one halogen radical, those halogen radicals may be identical or different (unless otherwise stated).

The term “acyl” includes alkanoyl containing from 1 to about 20 carbon atoms, preferably 1 to 6 carbon atoms, and may be straight-chain or branched. These groups include, for example, formyl, acetyl, propionyl, butyryl, isobutyryl, tertiary butyryl, pentanoyl and isomers of pentanoyl, and hexanoyl and isomers of hexanoyl.

The terms “electron-withdrawing” and “electron donating” refer to the ability of a substituent to withdraw or donate electrons, respectively, relative to that of hydrogen if the hydrogen atom occupied the same position in the molecule. These terms are well understood by one skilled in the art and are discussed in Advanced Organic Chemistry, by J. March, John Wiley and Sons, New York, N.Y., pp. 16-18 (1985) and the discussion therein is incorporated by reference. Electron withdrawing groups include halo, including bromo, fluoro, chloro, iodo and the like; nitro, carboxy, lower alkenyl, lower alkynyl, formyl, carboxyamido, aryl, quaternary ammonium, haloalkyl such as trifluoromethyl, aryl lower alkanoyl, carbalkoxy and the like. Electron donating groups include such groups as hydroxy, lower alkoxy, including methoxy, ethoxy and the like; lower alkyl, such as methyl, ethyl, and the like; amino, lower alkylamino, di(lower alkyl)amino, aryloxy such as phenoxy, mercapto, lower alkylthio, lower alkylmercapto, disulfide (lower alkyldithio) and the like. One of ordinary skill in the art will appreciate that some of the aforesaid substituents may be considered to be electron donating or electron withdrawing under different chemical conditions. Moreover, the present invention contemplates any combination of substituents selected from the above-identified groups.

As employed herein, a heterocyclic group contains at least one sulfur, nitrogen or oxygen ring atom, but also may include several of said atoms in the ring. The heterocyclic groups contemplated by the present invention include heteroaromatics and saturated and partially saturated heterocyclic compounds. Heterocyclics may be monocyclic, bicyclic, tricyclic or polycyclic and can be fused rings. They may preferably contain up to 18 ring atoms and up to a total of 17 ring carbon atoms and a total of up to 25 carbon atoms. Heterocyclics also include the so-called benzoheterocyclics. Representative heterocyclics include furyl, thienyl, pyrazolyl, pyrrolyl, methylpyrrolyl, imidazolyl, indolyl, thiazolyl, oxazolyl, isothiazolyl, isoxazolyl, piperidyl, pyrrolinyl, piperazinyl, quinolyl, triazolyl, tetrazolyl, isoquinolyl, benzofuryl, benzothienyl, morpholinyl, benzoxazolyl, tetrahydrofuryl, pyranyl, indazolyl, purinyl, indolinyl, pyrazolindinyl, imidazolinyl, imadazolindinyl, pyrrolidinyl, furazanyl, N-methylindolyl, methylfuryl, pyridazinyl, pyrimidinyl, pyrazinyl, pyridyl, epoxy, aziridino, oxetanyl, azetidinyl, the N-oxides of the nitrogen containing heterocycles, such as the N-oxides of pyridyl, pyrazinyl, and pyrimidinyl and the like. Heterocyclic groups may be unsubstituted or mono or polysubstituted with electron withdrawing or/and electron donating groups.

The preferred heterocyclics are thienyl, furyl, pyrrolyl, benzofuryl, benzothienyl, indolyl, methylpyrrolyl, morpholinyl, pyridyl, pyrazinyl, imidazolyl, pyrimidinyl, or pyridazinyl. The preferred heterocyclic is a 5 or 6-membered heterocyclic compound. The especially preferred heterocyclic is furyl, pyridyl, pyrazinyl, imidazolyl, pyrimidinyl, or pyridazinyl. The most preferred heterocyclics are furyl and pyridyl.

The preferred compounds of Formula (Ib) are those wherein n is 1, but di-(n=2), tri-(n=3) and tetrapeptides (n=4) are also contemplated to be within the scope of the invention.

The preferred R groups are aryl lower alkyl, especially benzyl, more especially those wherein the phenyl ring thereof is unsubstituted or substituted with electron donating groups or/and electron withdrawing groups, such as halo (e.g., F).

The preferred R₁ is H or lower alkyl. The most preferred R₁ group is methyl.

The preferred electron donating substituents or/and electron withdrawing substituents are halo, nitro, alkanoyl, formyl, arylalkanoyl, aryloyl, carboxyl, carbalkoxy, carboxamido, cyano, sulfonyl, sulfoxide, heterocyclic, guanidine, quaternary ammonium, lower alkenyl, lower alkynyl, sulfonium salts, hydroxy, lower alkoxy, lower alkyl, amino, lower alkylamino, di(loweralkyl)amino, amino lower alkyl, mercapto, mercaptoalkyl, alkylthio, and alkyldithio. The term “sulfide” encompasses mercapto, mercapto alkyl and alkylthio, while the term disulfide encompasses alkylthio. Especially preferred electron donating or/and electron withdrawing groups are halo or lower alkoxy, most preferred are fluoro or methoxy. These preferred substituents may be present in any one of the groups in Formula (Ib), (IIb) or (III), e.g., R, R₁, R₂, R₃, R₄, R₅, R₆, R′₆, R₇ or R₈ as defined herein.

The Z-Y groups representative of R₂ and R₃ include hydroxy; alkoxy, such as methoxy and ethoxy; aryloxy, such as phenoxy; thioalkoxy, such as thiomethoxy and thioethoxy; thioaryloxy such as thiophenoxy; amino; alkylamino, such as methylamino and ethylamino; arylamino, such as anilino; lower dialkylamino, such as dimethylamino; trialkylammonium salt; hydrazino; alkylhydrazino and arylhydrazino, such as N-methylhydrazino and N-phenylhydrazino; carbalkoxy hydrazino; aralkoxycarbonyl hydrazino; aryloxycarbonyl hydrazino; hydroxylamino, such as N-hydroxylamino (—NH—OH); lower alkoxyamino (NHOR₁₈ wherein R₁₈ is lower alkyl), N-lower alkylhydroxylamino (N(R₁₈)OH wherein R₁₈ is lower alkyl), N-lower alkyl-O-lower alkylhydroxylamino (N(R₁₈)OR₁₉ wherein R₁₈ and R₁₉ are independently lower alkyl), and o-hydroxylamino (—O—NH₂); alkylamido, such as acetamido; trifluoroacetamido; lower alkoxyamino, (e.g., NH(OCH₃); and heterocyclicamino, such as pyrazoylamino.

The preferred heterocyclic groups representative of R₂ and R₃ are monocyclic 5- or 6-membered heterocyclic moieties of the formula: 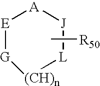
or those corresponding partially or fully saturated form thereof wherein n is 0 or 1; and R₅₀ is H or an electron withdrawing group or electron donating group; A, E, L, J and G are independently CH, or a heteroatom selected from the group consisting of N, O and S; but when n is 0, G is CH, or a heteroatom selected from the group consisting of NH, O and S with the proviso that at most two of A, E, L, J and G are heteroatoms.

When n is 0, the above heteroaromatic moiety is a five membered ring, while if n is 1, the heterocyclic moiety is a six-membered monocyclic heterocyclic moiety. The preferred heterocyclic moieties are those aforementioned heterocyclics which are monocyclic.

If the ring depicted hereinabove contains a nitrogen ring atom, then the N-oxide forms are also contemplated to be within the scope of the invention.

When R₂ or R₃ is a heterocyclic of the above formula, it may be bonded to the main chain by a ring carbon atom. When n is 0, R₂ or R₃ may additionally be bonded to the main chain by a nitrogen ring atom.

Other preferred moieties of R₂ and R₃ are hydrogen, aryl, e.g., phenyl; arylalkyl, e.g., benzyl; and alkyl.

It is to be understood that the preferred groups of R₂ and R₃ may be unsubstituted or mono- or polysubstituted with electron donating or/and electron withdrawing groups. It is preferred that R₂ and R₃ are independently hydrogen; lower alkyl, which is either unsubstituted or substituted with electron withdrawing groups or/and electron donating groups such as lower alkoxy (e.g., methoxy, ethoxy, and the like); N-hydroxylamino, N-lower-alkylhydroxyamino, N-lower-alkyl-O-lower-alkyl and alkylhydroxylamino.

It is preferred that one of R₂ and R₃ is hydrogen.

It is preferred that n is one.

It is more preferred that n=1 and one of R₂ and R₃ is hydrogen. It is especially preferred that in this embodiment, R₂ is hydrogen and R₃ is lower alkyl or Z-Y where Z is O, NR₄ or PR₄, and Y is hydrogen or lower alkyl; or Z-Y is NR₄NR₅R₇, NR₄OR₅, ONR₄R₇,

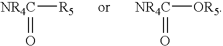

In another especially preferred embodiment, n=1, R₂ is hydrogen and R₃ is lower alkyl which may be substituted or unsubstituted with an electron donating or electron withdrawing group, NR₄OR₅, or ONR₄R₇.

In yet another especially preferred embodiment, n=1, R₂ is hydrogen and R₃ is lower alkyl which is unsubstituted or substituted with hydroxy or loweralkoxy, NR₄OR₅ or ONR₄R₇, wherein R₄, R₅ and R₇ are independently hydrogen or lower alkyl, R is aryl lower alkyl, which aryl group may be unsubstituted or substituted with an electron withdrawing group, and R₁ is lower alkyl. In this embodiment it is most preferred that aryl is phenyl, which is unsubstituted or substituted with halo.

It is preferred that R₂ is hydrogen and R₃ is hydrogen, an alkyl group which is unsubstituted or substituted by at least an electron donating or electron withdrawing group or Z-Y. In this preferred embodiment, it is more preferred that R₃ is hydrogen, an alkyl group such as methyl, which is unsubstituted or substituted by an electron donating group, or NR₄OR₅ or ONR₄R₇, wherein R₄, R₅ and R₇ are independently hydrogen or lower alkyl. It is preferred that the electron donating group is lower alkoxy, and especially methoxy or ethoxy.

It is preferred that R₂ and R₃ are independently hydrogen, lower alkyl, or Z-Y; Z is O, NR₄ or PR₄; Y is hydrogen or lower alkyl; or ZY is NR₄R₅R₇, NR₄OR₅, ONR₄R₇,

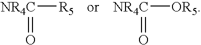

It is also preferred that R is aryl lower alkyl. The most preferred aryl for R is phenyl. The most preferred R group is benzyl. In a preferred embodiment, the aryl group may be unsubstituted or substituted with an electron donating or electron withdrawing group. If the aryl ring in R is substituted, it is most preferred that it is substituted with an electron withdrawing group, especially on the aryl ring. The most preferred electron withdrawing group for R is halo, especially fluoro.

The preferred R₁ is lower alkyl, especially methyl.

It is more preferred that R is aryl lower alkyl and R₁ is lower alkyl.

Further preferred compounds are compounds of Formula (Ib) wherein n is 1; R₂ is hydrogen; R₃ is hydrogen, a lower alkyl group, especially methyl which is substituted by an electron donating or electron withdrawing group, or Z-Y; R is aryl or aryl lower alkyl, such as benzyl, wherein the aryl group is unsubstituted or substituted with an electron donating or electron withdrawing group; and RI is lower alkyl. In this embodiment, it is more preferred that R₃ is hydrogen, a lower alkyl group, especially methyl, which may be substituted by an electron donating group, such as lower alkoxy, (e.g., methoxy, ethoxy and the like), NR₄OR₅ or ONR₄R₇ wherein these groups are as defined hereinabove.

In various embodiments, the compounds utilized are those of Formula (IV): 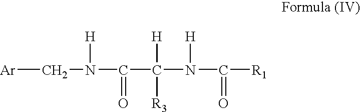
wherein:
Ar is aryl, especially phenyl, which is unsubstituted or substituted with at least one electron donating group or electron withdrawing group, especially halo;
R₁ is lower alkyl, especially containing 1-3 carbon atoms; and
R₃ is as defined herein, but especially hydrogen or lower alkyl, which is unsubstituted or substituted by at least one electron donating group or electron withdrawing group or Z-Y.

It is even more preferred that R₃ is, in this embodiment, hydrogen or an alkyl group which is unsubstituted or substituted by an electron donating group, NR₄OR₅ or ONR₄R₇. It is most preferred that R₃ is CH₂-Q, wherein Q is lower alkoxy, especially containing 1-3 carbon atoms; or NR₄OR₅ or ONR₄R₇ wherein R₄ is hydrogen or alkyl containing 1-3 carbon atoms, R₅ is hydrogen or alkyl containing 1-3 carbon atoms, and R₇ is hydrogen or alkyl containing 1-3 carbon atoms.

The most preferred R₁ is CH₃. The most preferred R₃ is CH₂-Q, wherein Q is methoxy.

The most preferred aryl is phenyl. The most preferred halo is fluoro.

It is to be understood that the various combinations and permutations of the Markush groups of R₁, R₂, R₃, R and n described herein are contemplated to be within the scope of the present invention. Moreover, the present invention also encompasses compounds and compositions which contain one or more elements of each of the Markush groupings in R₁, R₂, R₃, n and R and the various combinations thereof. Thus, for example, the present invention contemplates that R₁ may be one or more of the substituents listed hereinabove in combination with any and all of the substituents of R₂, R₃, and R with respect to each value of n.

The compounds utilized in the present invention may contain one or more asymmetric carbons and may exist in racemic and optically active forms. The configuration around each asymmetric carbon can be either the D or L form. It is well known in the art that the configuration around a chiral carbon atoms can also be described as R or S in the Cahn-Prelog-Ingold nomenclature system. All of the various configurations around each asymmetric carbon, including the various enantiomers and diastereomers as well as racemic mixtures and mixtures of enantiomers, diastereomers or both are contemplated by the present invention.

In the principal chain, there exists asymmetry at the carbon atom to which the groups R₂ and R₃ are attached. When n is 1, a compound useful herein is of the formula 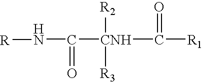
wherein R, R₁, R₂, R₃, R₄, R₅, R₆, R′₆, R₇, R₈, R₅₀ Z and Y are as defined previously.

As used herein, the term configuration shall refer to the configuration around the carbon atom to which R₂ and R₃ are attached, even though other chiral centers may be present in the molecule. Therefore, when referring to a particular configuration, such as D or L, it is to be understood to mean the D or L stereoisomer at the carbon atom to which R₂ and R₃ are attached. However, it also includes all possible enantiomers and diastereomers at other chiral centers, if any, present in the compound.

The compounds of the present invention are directed to all the optical isomers, i.e., the compounds of the present invention are either the L-stereoisomer or the D-stereoisomer (at the carbon atom to which R₂ and R₃ are attached). These stereoisomers may be found in mixtures of the L and D stereoisomer, e.g., racemic mixtures. The D stereoisomer is preferred. In lacosamide, the D stereoisomer corresponds to the R enantiomer according to the R,S terminology.

Depending upon the substituents, the present compounds may form addition salts as well. All of these forms are contemplated to be within the scope of this invention including mixtures of the stereoisomeric forms.

The manufacture of the utilized compounds is described in U.S. Pat. Nos. 5,378,729 and 5,773,475, the contents of which are incorporated by reference.

The compounds utilized in the present invention are useful as such as depicted in Formula (III) or can be employed in the form of salts by the presence of the free amino group. Thus, the compounds of Formula (III) form salts with a wide variety of acids, inorganic and organic, including pharmaceutically acceptable acids. The salts with therapeutically acceptable acids are useful in the preparation of formulation where enhanced water solubility is most advantageous.

These pharmaceutically acceptable salts have also therapeutic efficacy. These salts include salts of inorganic acids such as hydrochloric, hydroiodic, hydrobromic, phosphoric, metaphosphoric, nitric and sulfuric acids as well as salts of organic acids, such as tartaric, acetic, citric, malic, benzoic, perchloric, glycolic, gluconic, succinic, arylsulfonic (e.g., p-toluene sulfonic acids, benzenesulfonic), phosphoric and malonic acids and the like.

The present invention is further directed to a method for the prevention, alleviation or/and treatment of psychosis in an add-on therapy to at least one psychosis-treating compound by administration of at least one compound of Formula (III) to a subject in need thereof.

In a preferred embodiment, the method of the present invention comprises the administration of at least one psychosis-treating compound, and the administration of at least one compound of Formula (III) in an add-on therapy in addition to the at least one psychosis-treating compound, to a subject in need thereof.

The compounds according to Formula (III) may also be useful in an add-on therapy to at least one psychosis-treating compound for the treatment of psychosis not associated with schizophrenia which is treated by psychosis-treating compounds. Therefore, yet another subject of the present invention is an add-on therapy, by administration of at least one compound of Formula (III) in addition to at least one psychosis-treating compound, for the prevention, alleviation or treatment of psychosis associated with, for example, bipolar disorder, autism, Alzheimer's disease, attention deficit hyperactivity disorder, drug abuse, alcohol abuse, affective disorders, dyskinesias, dyskinesia-related disorders, dementia, mental retardation, polydipsia/hyponatremia, severe personality disorder, acute episodes of mania, obsessive compulsive disorder, intractable chronic insomnia, Huntington's disease, Tourette's syndrome, Parkinson's disease, and dopaminergic therapy of Parkinson's disease.

The compounds according to Formula (III) may also be used for the preparation of a pharmaceutical composition useful for the prevention, alleviation or/and treatment in an add-on therapy with at least one psychosis-treating compound, for the prevention, alleviation or treatment of psychosis associated with, for example, bipolar disorder, autism, Alzheimer's disease, attention deficit hyperactivity disorder, drug abuse, alcohol abuse, affective disorders, dyskinesias, dyskinesia-related disorders, dementia, mental retardation, polydipsia/hyponatremia, severe personality disorder, acute episodes of mania, obsessive compulsive disorder, intractable chronic insomnia, Huntington's disease, Tourette's syndrome, Parkinson's disease, and dopaminergic therapy of Parkinson's disease.

It is preferred that the compounds utilized in the present invention are used in therapeutically effective amounts.

The physician will determine the dosage of the present therapeutic agents which will be most suitable and it will vary with the form of administration and the particular compound chosen, and furthermore, it will vary with the patient under treatment, the age of the patient, the type of malady being treated. He will generally wish to initiate treatment with small dosages substantially less than the optimum dose of the compound and increase the dosage by small increments until the optimum effect under the circumstances is reached. When the composition is administered orally, larger quantities of the active agent will be required to produce the same effect as a smaller quantity given parenterally. The compounds are useful in the same manner as comparable therapeutic agents and the dosage level is of the same order of magnitude as is generally employed with these other therapeutic agents.

Typical doses of psychosis-treating compounds administered to a subject in need thereof in the add-on therapy of the present invention are: olanzapine, about 5 to about 20 mg/day; clozapine, about 100 to about 900 mg/day; quetiapine, about 100 to about 800 mg/day; risperidone, about 1 to about 16 mg/day; aripiprazole, about 3 to about 30 mg/day; ziprasidone, about 40 to about 160 mg/day; sulpiride, about 300 to about 1600 mg/day; amisulpride, about 50 to about 1200 mg/day; zotepine, about 75 to about 450 mg/day. Such doses may also be included in a pharmaceutical composition of the present invention, or may be used for the preparation of a pharmaceutical composition of the present invention.

The physician is able to determine the administration route of the at least one psychosis-treating compound, which may be the same as or different from the administration route of the at least one compound of Formula (III). Typical administration routes of the at least one psychosis-treating compound are oral (p.o.), intravenous (i.v.), intramuscular (i.m.), intrathecal or subcutaneous routes. Oral or intravenous administration is preferred.

In a preferred embodiment, the at least one compound of Formula (III) of the present invention is administered in an amount ranging from about 1 mg to about 10 mg per kilogram of body weight per day. This dosage regimen may be adjusted by the physician to provide the optimum therapeutic response. Patients in need thereof may be treated with doses of the compound of the present invention of at least about 50 mg/day, preferably at least about 100 mg, more preferably at least about 200 mg/day, more preferably at least about 300 mg/day and most preferably at least about 400 mg/day. Generally, a patient in need thereof may be treated with doses at a maximum of about 6 g/day, more preferably a maximum of about 1 g/day and most preferably a maximum of about 600 mg/day. In some cases, however, higher or lower doses may be needed.

In another preferred embodiment, the daily doses are increased until a predetermined daily dose is reached which is maintained during the further treatment.

In yet another preferred embodiment, several divided doses may be administered daily. For example, three doses per day may be administered, preferably two doses per day. It is more preferred to administer a single dose per day.

Doses expressed herein on a daily basis, for example in mg/day, are not to be interpreted as requiring a once-a-day frequency of administration. For example, a dose of 300 mg/day can be given as 100 mg three times a day, or as 600 mg every second day.

In yet another preferred embodiment, an amount of the at least one compound according to Formula (III) may be administered which results in a plasma concentration of about 0.1 to about 15 μg/ml (trough) and about 5 to about 18.5 μg/ml (peak), calculated as an average over a plurality of treated subjects.

The compounds of Formula (III) may be administered in a convenient manner, such as by oral, intravenous, intramuscular, intrathecal or subcutaneous routes. Oral or intravenous administration is preferred.

The pharmaceutical composition of the present invention may be prepared for the treatment regimen as described above, in particular for the treatment with doses as described above, to effect plasma concentrations as described above, for administration periods or administration routes as specified in the embodiments of the present invention as described above.

Pharmaceutical compositions of the present invention comprising the at least one psychosis-treating compound can be formulated with common carriers, diluents and/or auxiliary substances known to a person skilled in the art. In the case of a separate dose form, the first composition comprising at least one psychosis-treating compound and the second composition comprising at least one compound of Formula (III) may comprise carriers, diluents or/and auxiliary substances which are independent from each other, identical or different in the first composition and the second composition.

The compounds of Formula (III) may be orally administered, for example, with an inert diluent or with an assimilable edible carrier, or it may be enclosed in hard or soft shell gelatin capsules, or it may be compressed into tablets, or it may be incorporated directly into food or the diet. For oral therapeutic administration, an active compound of Formula (III) may be incorporated with excipients and used in the form of ingestible tablets, buccal tablets, troches, capsules, elixirs, suspensions, syrups, wafers, and the like. Such compositions and preparations should typically contain at least 1% of active compound of Formula (III). The percentage of active compound in the compositions and preparations may, of course, be varied and may conveniently be about 5% to about 80% of the weight of the unit. The amount of active compound of Formula (III) in such therapeutically useful compositions is such that a suitable dosage will be obtained. Preferred compositions or preparations according to the present invention contain between about 10 mg and about 6 g active compound of Formula (III).

The tablets, troches, pills, capsules and the like may also contain the following: a binder such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, lactose or saccharin may be added or a flavoring agent such as peppermint, oil of wintergreen, or cherry flavoring. When the dosage unit form is a capsule, it may contain, in addition to materials of the above type, a liquid carrier.

Various other materials may be present as coatings or otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring such as cherry or orange flavor. Any material used in preparing any dosage unit form should be pharmaceutically pure and substantially non-toxic in the amounts employed. In addition, the active compound may be incorporated into sustained-release preparations and formulations. For example, sustained release dosage forms are contemplated wherein the active ingredient is bound to an ion exchange resin which, optionally, can be coated with a diffusion barrier coating to modify the release properties of the resin.

The active compound may also be administered parenterally or intraperitoneally. Dispersions can also be prepared in glycerol, liquid, polyethylene glycols, and mixtures thereof and in oils. Under ordinary conditions of storage and use, these preparations may contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, liquid polyethylene glycol, and the like), suitable mixtures thereof, and vegetable oils. The proper fluidity can be maintained, for example, by the use of a coating such as lecithin, by the maintenance of the required particle size in the case of dispersions and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying the freeze-drying technique plus any additional desired ingredient from previously sterile-filtered solution thereof.

As used herein, “pharmaceutically acceptable carrier” includes any and all solvents, dispersion media, coatings, antibacterial and antifungal agent, isotonic and absorption delaying agents for pharmaceutical active substances as well known in the art. Except insofar as any conventional media or agent is incompatible with the active ingredient, its use in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specifics for the novel dosage unit forms of the invention are dictated by and directly dependent on (a) the unique characteristics of the active material an the particular therapeutic effect to be achieved, and (b) the limitations inherent in the art of compounding such as active material for the treatment of disease in living subjects having a diseased condition in which bodily health is impaired as herein disclosed in detail.

The at least one compound of Formula (III) is compounded for convenient and effective administration in effective amounts with a suitable pharmaceutically acceptable carrier in dosage unit form as hereinbefore described. A unit dosage form can, for example, contain the compound in amounts ranging from about 10 mg to about 6 g. Expressed in proportions, the active compound is generally present in the carrier in an amount from about 1 to about 750 mg/ml of carrier. In the case of compositions containing supplementary active ingredients, the dosages are determined by reference to the usual dose and manner of administration of the said ingredients.

The compounds of the present invention are administered to a patient suffering from the aforementioned type of disorder in an effective amount. These amounts are equivalent to the therapeutically effective amounts described hereinabove.

### EXAMPLE

The following example shows the properties of lacosamide, formerly called harkoseride, or SPM 927, potentiating the prepulse inhibition of the antipsychotic agent clozapine in mice when administered together. The used substance was lacosamide. The standard chemical nomenclature is (R)-2-acetamide-N-benzyl-3-methoxypropionamide.

### EXAMPLE

### The Effects of Lacosamide Alone and in Combination with Clozapine in an Animal Model for Schizophrenia

Some anticonvulsants have proven pre-clinical and/or clinical efficacy as add-on therapy to antipsychotics for schizophrenia. Lacosamide was tested in a simple animal model with predictive validity for schizophrenia (i.e. prepulse inhibition (PPI) of the acoustic startle reflex). Lacosamide was tested alone and in combination with the atypical antipsychotic agent clozapine.

The acoustic startle is an unconditioned reflex response to an external auditory stimulation. PPI refers to the reduction in the startle response caused by the presentation of a low-intensity auditory stimulus prior to the startle stimulus. The PPI paradigm is the choice for the study of schizophrenia and antipsychotic action due to the similarities between the results from human and rodent studies. Antipsychotic agents, such as clozapine, result in a dose-dependent increase in PPI in mice. Thus, an increase of PPI in normal mice may be indicative of antipsychotic efficacy.

C57BL/6J mice obtained from the Jackson Laboratory, Bar Harbor, Me. were received at the age of 6 weeks and were assigned unique identification numbers (tail marked). Animals were housed 4 per cage in polycarbonate cages with filter tops and acclimated for 7 days. All animals were examined, handled, and weighed prior to initiation of the study to assure adequate health and suitability and to minimize non-specific stress associated with manipulation.

During the course of the study, 12/12 light/dark cycles and a room temperature of 20 to 23° C. were maintained with a relative humidity maintained around 50%. Chow and water were provided ad libitum for the duration of the study. Each mouse was randomly assigned across the treatment groups and balanced by cage numbers. The test was performed during the animal's light cycle phase. Twelve animals were used in each treatment group.

Lacosamide was dissolved in saline (0.9% NaCl in sterile H₂O). Clozapine was dissolved in 1% Tween 80 in sterile H₂O at a final pH of 6.0. All compounds were administered intraperitoneally (i.p.) (volume injection: 10 ml/kg). Lacosamide was administered at doses of 1, 10 and 30 mg/kg and clozapine at a dose of 3 mg/kg. Doses are expressed as mg of salt. Control mice were administered saline (vehicle 1, VEH 1) or 1% Tween 80 (vehicle 2, VEH 2) at a pH of 6.0. The drug treatments were balanced across days and the animals only used once. All tests were conducted blind. All compounds were administered 30 min prior to testing.

Animals were placed in the PPI chambers (Med Associates) for a 5 min session of white noise (70 dB) habituation. After the acclimation period the test session was automatically started. The session started with an habituation block of 6 presentations of the startle stimulus alone, followed by 10 PPI blocks of 6 different types of trials. Trial types are: null (no stimuli), startle (120 dB), startle plus prepulse (4, 8 and 12 dB over background noise i.e. 74, 78 or 82 dB) and prepulse alone (82 dB). Trial types were presented at random within each block. Each trial started with a 50 ms null period during which baseline movements are recorded. There was a subsequent 20 ms period during which prepulse stimuli were presented and responses to the prepulse measured. After further 100 ms the startle stimuli were presented for 40 ms and responses recorded for 100 ms from startle onset. Responses were sampled every ms. The inter-trial interval was variable with an average of 15 s (range from 10 to 20 s). In startle alone trials the basic auditory startle is measured and in prepulse plus startle trials the amount of inhibition of the normal startle is determined and expressed as a percentage of the basic startle response (from startle alone trials), excluding the startle response of the first habituation block. Eight animals were tested at one time.

Twelve animals were tested in each group. A total of 108 mice were tested using following design:

VEH1/VEH2
lacosamide [3 mg/kg]/VEH2
lacosamide [10 mg/kg]/VEH2
lacosamide [30 mg/kg]/VEH2
VEH1/clozapine [3 mg/kg]
lacosamide [3 mg/kg]/clozapine [3 mg/kg]
lacosamide [10 mg/kg]/clozapine [3 mg/kg]
lacosamide [30 mg/kg]/clozapine [3 mg/kg]

A two-way ANOVA (analysis of variance) with treatments as independent factor and pre-pulse intensities as dependent factor (i.e. repeated measure) was performed. This was followed by the post-hoc Newman Keuls test where indicated. A p<0.05 was considered significant. The Newman Keuls test allows for the comparison of two independent samples.

Animals injected with clozapine (3 mg/kg) showed a significant improvement in prepulse inhibition in comparison to animals receiving vehicle (p<0.05, Table 1). When administered alone, lacosamide did not have any significant effect on prepulse inhibition at any dose tested, when compared to vehicle. The administration of lacosamide (at 30 mg/kg but not at 3 or 10 mg/kg) in combination with clozapine however, did potentiate the effect of clozapine alone on prepulse inhibition, i.e., the prepulse inhibition was significantly higher than the effect of clozapine alone (p<0.05).

The data obtained provide a basis for clinical trials in humans using lacosamide as an add-on therapy for the treatment of schizophrenia.

TABLE 1
Effects of lacosamide alone and in combination with clozapine
on prepulse inhibition of the acoustic startle response (PPI)

| Treatment | Mean PPI |
|---|---|
| VEH 1/VEH 2 | 29 ± 3 |
| VEH 1/clozapine [3 mg/kg] | 45 ± 2* |
| lacosamide [3 mg/kg]/VEH 2 | 29 ± 3 |
| lacosamide [10 mg/kg]/VEH 2 | 34 ± 3 |
| lacosamide [30 mg/kg]/VEH 2 | 27 ± 2 |
| lacosamide [3 mg/kg]/clozapine [3 mg/kg] | 43 ± 2* |
| lacosamide [10 mg/kg]/clozapine [3 mg/kg] | 45 ± 2* |
| lacosamide [30 mg/kg]/clozapine [3 mg/kg] | 57 ± 5*, # |

*p < 0.05 for comparison to VEH/VEH group
# p < 0.05 for comparison to VEH/CLOZ group

What is claimed is:

1. A therapeutic combination comprising a first agent and a second agent, wherein the first agent comprises at least one psychosis-treating compound and the second agent comprises at least one compound, or a pharmaceutically acceptable salt thereof, according to Formula (III): 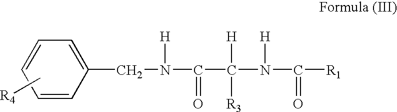
wherein:
R₄ is one or more substituents independently selected from the group consisting of hydrogen, halo, alkyl, alkenyl, alkynyl, nitro, carboxy, formyl, carboxyamido, aryl, quaternary ammonium, haloalkyl, aryl alkanoyl, hydroxy, alkoxy, amino, alkylamino, dialkylamino, aryloxy, mercapto, alkylthio, alkylmercapto and disulfide;
R₃ is selected from the group consisting of hydrogen, alkyl, alkoxy, alkoxyalkyl, aryl, N-alkoxy-N-alkylamino and N-alkoxyamino; and
R₁ is alkyl.

2. The combination of claim 1, wherein, in the at least one compound of Formula (III):
R₄ is one or more substituents independently selected from the group consisting of hydrogen and halo;
R₃ is selected from the group consisting of lower-alkoxy-lower-alkyl, aryl, N-lower-alkoxy-N-lower-alkylamino, and N-lower-alkoxyamino; and
R₁ is lower alkyl.

3. The combination of claim 1, wherein, in the at least one compound of Formula (III):
R₄ is hydrogen or fluoro;
R₃ is selected from the group consisting of methoxymethyl, phenyl, N-methoxy-N-methylamino, and N-methoxyamino; and
R₁ is methyl.

4. The combination of claim 1, wherein the at least one compound of Formula (III) is selected from the group consisting of
(R)-2-acetamido-N-benzyl-3-methoxy-propionamide;
(R)-2-acetamido-N-benzyl-3-ethoxy-propionamide;
O-methyl-N-acetyl-D-serine-m-fluorobenzylamide;
O-methyl-N-acetyl-D-serine-p-fluorobenzylamide;
N-acetyl-D-phenylglycinebenzylamide;
D-1,2-(N,O-dimethylhydroxylamino)-2-acetamide acetic acid benzylamide; and
D-1,2-(O-methylhydroxylamino)-2-acetamide acetic acid benzylamide.

5. The combination of claim 1, wherein in the at least one compound of Formula (III):
R4 is one or more substituents independently selected from the group consisting of hydrogen and halo;
R₃ is lower-alkoxy-lower-alkyl; and
R₁ is lower alkyl.

6. The combination of claim 1, wherein in the at least one compound of Formula (III):
R₄ is hydrogen;
R₃ is methoxymethyl; and
R₁ is methyl.

7. The combination of claim 1, wherein the second agent is substantially enantiopure.

8. The combination of claim 1, wherein the at least one compound of Formula (III) is lacosamide.

9. The combination of claim 1, wherein the at least one psychosis-treating compound is selected from the group consisting of tricyclic antipsychotics, phenothiazines, thioxanthenes, butyrophenones, dihydroindolones and dibenzoxazepines.

10. The combination of claim 1, wherein the at least one psychosis-treating compound is selected from the group consisting of amisulpride, aripiprazole, biriperone, bromperidol, carpipramine, clocapramine, clorotepine, clozapine, isofloxythepin, melperone, mosapramine, nemonapride, olanzapine, penfluridol, perospirone, pimazide, pipotiazine palmitate, quetiapine fumarate, risperidone, sulpiride, sultopride, tiapride, timiperone, ziprasidone, zotepine, zuclopenthixol, zuclopenthixol acetate and zuclopenthixol decanoate.

11. The combination of claim 1, wherein the at least one psychosis-treating compound is selected from the group consisting of clozapine, risperidone, aripiprazole, quetiapine, olanzapine, sulpiride, amisulpride, ziprasidone and zotepine.

12. The combination of claim 1, wherein the at least one psychosis-treating compound is clozapine.

13. The combination of claim 1, wherein the first agent comprises clozapine and the second agent comprises lacosamide.

14. A co-therapy method for treating psychosis, comprising administering to a subject a first amount of a first agent comprising at least one psychosis-treating compound and a second amount of a second agent comprising at least one compound, or a pharmaceutically acceptable salt thereof, according to Formula (III): 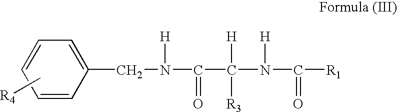
wherein:
R₄ is one or more substituents independently selected from the group consisting of hydrogen, halo, alkyl, alkenyl, alkynyl, nitro, carboxy, formyl, carboxyamido, aryl, quaternary ammonium, haloalkyl, aryl alkanoyl, hydroxy, alkoxy, amino, alkylamino, dialkylamino, aryloxy, mercapto, alkylthio, alkylmercapto, and disulfide;
R₃ is selected from the group consisting of hydrogen, alkyl, alkoxy, alkoxyalkyl, aryl, N-alkoxy-N-alkylamino, and N-alkoxyamino; and
R₁ is alkyl;
and wherein the first amount and second amount together comprise a therapeutically effective combination of the first agent and second agent.

15. The method of claim 14, wherein the first agent comprises clozapine and the second agent comprises lacosamide.

16. The method of claim 14, wherein the psychosis is associated with schizophrenia, bipolar disorder, autism, Alzheimer's disease, attention deficit hyperactivity disorder, drug abuse, alcohol abuse, affective disorders, dyskinesias, dyskinesia-related disorders, dementia, mental retardation, polydipsia/hyponatremia, severe personality disorder, acute episodes of mania, obsessive compulsive disorder, intractable chronic insomnia, Huntington's disease, Tourette's syndrome, Parkinson's disease, and dopaminergic therapy of Parkinson's disease.

17. The method of claim 14, wherein the psychosis is associated with schizophrenia.

18. The method of claim 14, wherein the first agent is administered at a dose of about 1 to about 400 mg/day and the second agent is administered at a dose of about 100 to about 6000 mg/day.

19. The method of claim 14, wherein the first agent is administered at a dose of about 1 to about 200 mg/day and the second agent is administered at a dose of about 200 to about 1000 mg/day.

20. The method of claim 14, wherein the first agent is administered at a dose of about 5 to about 100 mg/day and the second agent is administered at a dose of about 300 to about 600 mg/day.

21. The method of claim 14, wherein the second agent is administered according to a regime wherein daily doses are increased until a predetermined daily dose is reached which is maintained during further treatment.

22. The method of claim 14, wherein the second agent is administered in one to three doses per day.

23. The method of claim 15, wherein a peak plasma concentration of the second agent of about 0.1 to about 15 μg/ml, calculated as an average over a plurality of treated subjects, is obtained.

24. The method of claim 15, wherein the first and second agents are independently administered orally or intravenously.

25. A pharmaceutical composition comprising a first agent and a second agent, wherein the first agent comprises at least one psychosis-treating compound and the second agent comprises at least one compound, or a pharmaceutically acceptable salt thereof, according to Formula (III): 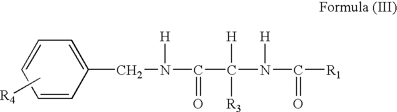
wherein:
R₄ is one or more substituents independently selected from the group consisting of hydrogen, halo, alkyl, alkenyl, alkynyl, nitro, carboxy, formyl, carboxyamido, aryl, quaternary ammonium, haloalkyl, aryl alkanoyl, hydroxy, alkoxy, amino, alkylamino, dialkylamino, aryloxy, mercapto, alkylthio, alkylmercapto, and disulfide;
R₃ is selected from the group consisting of hydrogen, alkyl, alkoxy, alkoxyalkyl, aryl, N-alkoxy-N-alkylamino, and N-alkoxyamino; and
R₁ is alkyl.
